(19) United States
(12) Patent Application Publication
(10) Pub. No.: US 2025/0242167 A1
(43) Pub. Date: Jul. 31, 2025

(54) CLOSED-LOOP NEUROSTIMULATION USING GLOBAL OPTIMIZATION-BASED TEMPORAL PREDICTION

(71) Applicant: Regents of the University of Minnesota, Minneapolis, MN (US)

(72) Inventors: Alexander OPITZ, Minneapolis, MN (US); Seyedsina SHIRINPOUR, Minneapolis, MN (US); Ivan ALEKSEICHUK, Minneapolis, MN (US)

(21) Appl. No.: 18/855,963

(22) PCT Filed: Apr. 10, 2023
(86) PCT No.: PCT/US2023/018071

§ 371 Date: Oct. 10, 2024

Related U.S. Application Data
(60) Provisional application No. 63/329,557, filed on Apr. 11, 2022.

(51) Int. Cl. A61N 2/00; A61N 2/02

(52) U.S. Cl. CPC A61N 2/006; A61N 2/02

(57) ABSTRACT

The delivery of neurostimulation to a subject using a closed-loop neurostimulation device (e.g., using a brain stimulation device to provide neurostimulation to a subject's brain) is controlled based on a global optimization-based temporal prediction framework. As a result, the brain stimulation device (e.g., a transcranial magnetic stimulation (“TMS”) device) is synchronized with the ongoing neural state (e.g., brain state) in real time. For instance, a brain recording is analyzed to extract the brain process of interest (e.g., frequency of brain oscillations) and used train a prediction algorithm. After that, a stimulation stage is implemented, in which the individual brain state is analyzed in real-time, the occurrence of the biomarkers (e.g., brain oscillation phase) is predicted, and the stimulation is triggered at the expected time.

### CROSS-REFERENCE TO RELATED APPLICATIONS

This application claims the benefit of U.S. Provisional Patent Application Ser. No. 63/329,557, filed on Apr. 11, 2022, and entitled “Closed-Loop Neurostimulation Using Global Optimization-Based Temporal Prediction,” which is herein incorporated by reference in its entirety.

### STATEMENT OF FEDERALLY SPONSORED RESEARCH

This invention was made with government support under NS109498 awarded by the National Institutes of Health. The government has certain rights in the invention.

### BACKGROUND

Neurostimulation (e.g., brain stimulation) encompasses a range of techniques used to invasively or non-invasively modulate neural activity (e.g., brain activity). Several brain stimulation modalities are FDA-cleared and used in the clinical treatment of psychiatric and neurological disorders. Although brain stimulation is increasingly used clinically, a substantial proportion of patients do not respond to the intervention. One promising approach to reduce the variability of outcomes and improve the effectiveness of brain stimulation is to individualize the timing of stimulation.

Brain activity is characterized by cyclical changes due to synchronization and desynchronization of local brain circuits. These brain oscillations can be measured through electrophysiological recordings such as electroencephalography (“EEG”) and categorized in a spectrum of frequency bands (e.g., delta, theta, alpha, beta, and gamma), corresponding to a distinct spatial distribution and function. The brain oscillations can provide information about the brain states vital to the functioning and excitability of brain networks. Importantly, brain oscillations differ from person to person, necessitating the need for individualization. Therefore, real-time state-dependent brain stimulation is a promising candidate for individualizing neuromodulation treatments.

### SUMMARY OF THE DISCLOSURE

The present disclosure addresses the aforementioned drawbacks by providing a method for controlling a brain stimulation device. The method includes selecting, by a computer system, a biomarker for controlling a delivery of neurostimulation with a neurostimulation device. The biomarker may include a neural oscillation phase, which in some instances may be a brain oscillation phase. Baseline neural signal data are accessed with the computer system, where the baseline neural signal data have been acquired from the brain of a subject. The baseline neural signal data are processed with the computer system to extract features from the baseline neural signal data corresponding to the selected biomarker. A predictive model of biomarker occurrences is then constructed based on the extracted features. Neural signal data are then received in real-time with the computer system, and the operation of the neurostimulation device is controlled by applying the neural signal data to the predictive model of biomarker occurrences, generating output as stimulation parameters that indicate time points at which to deliver neurostimulation, where the time points correspond to predicted occurrences of the biomarkers within the neural signal data.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1 illustrates an overview of phase-dependent stimulation using a global optimization-based temporal prediction framework. The nervous signal is recorded in real-time and sent to the processor to analyze the data. Initially, a short recording of the signal is collected and the machine learning algorithm extracts the informative features (prediction parameters). Then, based on these features, the next occurrence of the desired phase of the nervous signal is predicted in real-time and the stimulator is triggered at the right time. The user provides the desired oscillation and stimulation parameters, as well as system parameters which are hardware-dependent.

FIG. 2 illustrates a machine learning (offline) step of a global optimization-based temporal prediction framework. Nervous signal is first recorded to extract necessary prediction parameters. The spatial filter is calculated for the region of interest. Then, the threshold value for the epoch power is measured. Afterward, based on the calculated ground-truth (true phase), the optimal value for the rest of prediction parameters (projection distance, window length, padding length, edge length, and projection starting point) are computed in an iterative optimization process. Finally, all the predication parameters are outputted for use in the online step.

FIG. 3 illustrates optimization of prediction parameters. Given any input prediction parameters, the simulation module uses the ergodic recording and simulates the phase prediction process that occurs during the online step. Additionally, the simulation module compares the results of the simulation outcomes with the calculated true phase and outputs the performance error. Using a global optimization framework (e.g., a Bayesian optimization framework), the set of prediction parameters that would minimize the simulation performance error is iteratively determined. These parameters are then used for the online prediction step. Furthermore, if the user is interested in delivering a patterned stimulation, the algorithm adjusts the necessary parameters accordingly.

FIG. 4 illustrates an example online prediction paradigm. First, the nervous signal is preprocessed to extract the signal in the region and frequency band of interest. If the data epoch has high enough power, the starting point for projection is determined. Then, the target phase is projected from the starting point, based on the projection distance. Based on the predicted phase and the system criteria, a stimulation trigger is sent at the correct timing.

FIG. 5 is a flowchart of an example method for controlling the operation of a brain stimulation device based on a global optimization-based temporal predictive framework.

FIG. 6 is a block diagram of an example controller that can implement the methods described in the present disclosure.

### DETAILED DESCRIPTION

Described here are systems and methods for controlling the delivery of neurostimulation to a subject using a closed-loop neurostimulation device, such as using a brain stimulation device to provide neurostimulation to a subject's brain. As an example, the systems and methods described in the present disclosure enable synchronizing a brain stimulation device (e.g., a transcranial magnetic stimulation (“TMS”) device) with the ongoing brain state in real time. For instance, a short (e.g., 1-5 minute) individual brain recording is analyzed to extract the brain process of interest (e.g., a specific frequency band of brain oscillations) and used to train a prediction algorithm. After that, a stimulation stage is implemented, in which the individual brain state is analyzed in real-time, the occurrence of the biomarkers (e.g., brain oscillation phase) is predicted, and the stimulation is triggered at the expected time. This process is continually repeated until the total number of stimulation events is reached.

The systems and methods can be integrated with TMS systems or other neurostimulation systems for treating mental disorders or other neurological conditions. For example, TMS and its forms can be used for treatment-resistant depression, obsessive-compulsive disorder, and nicotine addiction. Advantageously, the systems and methods described in the present disclosure can improve the effectiveness of the stimulation procedure in all of these indications. The systems and methods can be utilized with other brain stimulation methods as well.

In general, a global optimization-based temporal prediction framework is provided for real-time detection of any desired brain oscillation phase or other biomarkers that can be detected from physiological measurement data, such as electroencephalography (“EEG”) data. In some embodiments, global optimization can be implemented using a Bayesian optimization, resulting in a Bayesian temporal prediction (“BTP”) framework. Neurostimulation is then provided to a subject based on the detected neural oscillation phase or other biomarker. Phase, which denotes the position of the time points in the oscillation cycle, is an important biomarker for brain excitability (or other nervous system excitability) and, therefore, can be used as an important feature for the individualization. In such an approach, the brain signals or other neural signals are measured and analyzed in real-time. The stimulation is delivered at the appropriate phase (relative to the ongoing neural oscillation of interest) to maximize a desired outcome. For instance, based on a detected neural oscillation phase, a time point at which to apply neurostimulation is determined and a neurostimulation device is controlled to provide stimulation at that time point.

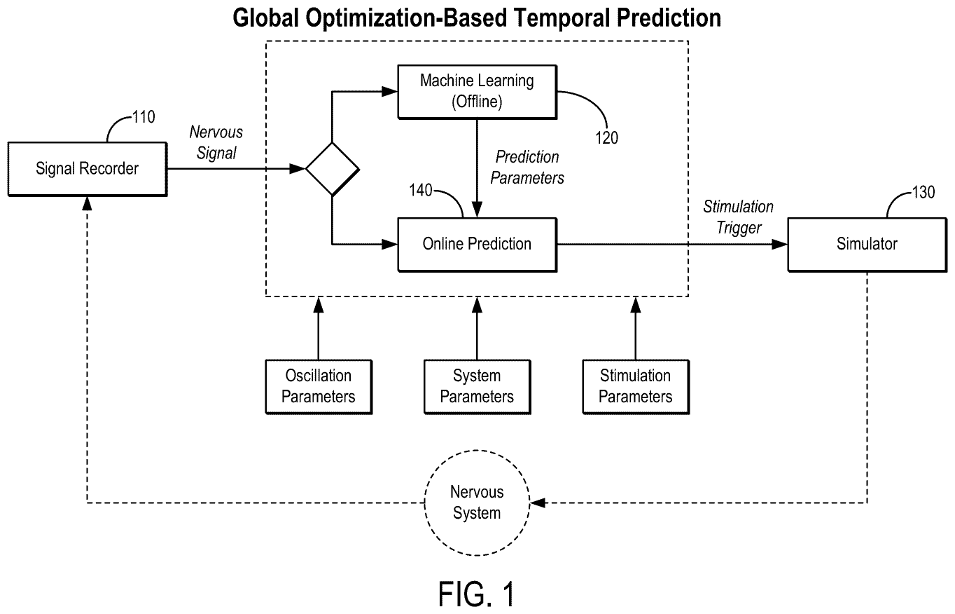

As illustrated in FIG. 1, the global optimization-based temporal prediction framework generally includes two steps or modules: a learning module 120 and an online prediction module 140. The global optimization-based temporal prediction framework takes neural signal data as an input, which may include recordings of brain signals. The neural signal data are acquired using a signal recorder 110, as an example, which may include an EEG device, another physiological measurement system, or the like. The global optimization-based temporal prediction framework can also take other inputs, such as oscillation parameters, system parameters, stimulation parameters, and the like.

In the learning module 120, the neural signal data is received from the signal recorder 110 and the global optimization-based temporal prediction framework automatically extracts the informative features needed for the phase prediction (i.e., prediction parameters). This process is performed for an individual user, thereby allowing for optimal stimulation parameters to be tailored to a particular individual.

In the online prediction module 140, the features extracted in the learning module 120 are used to predict the signal phase, or other biomarker, in real-time, which are subsequently used to control the operation of the neurostimulator 130 to deliver the stimulation at the correct time. The neurostimulator 130 may be an electrical stimulation device, a magnetic stimulation device, a sonic stimulation device, and/or a thermal stimulation device. As one non-limiting example, the neurostimulator 130 can be a TMS device. Unlike other methods, the global optimization-based temporal prediction framework does not aim to predict the whole signal and its corresponding instantaneous phase. Instead, the global optimization-based temporal prediction framework directly predicts the time point at which the next desired phase will occur, and thus the time point at which to provide neurostimulation. This reduces the computational demand and provides a robust personalized estimator for phase-dependent stimulation.

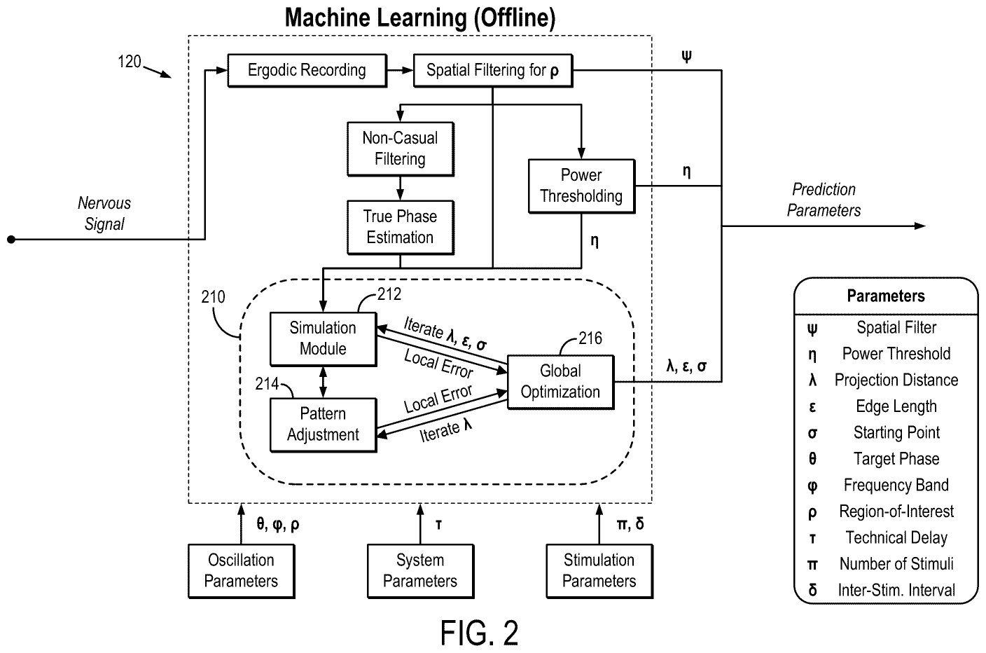

Referring now to FIG. 2, an example learning module 120 of a global optimization-based temporal prediction framework is illustrated. Neural signal data are received by the learning module 120. For example, the learning module 120 can be implemented as a software module, or the like, and the neural signal data can be received by a processor implementing the learning module 120, such as by receiving the neural signal data via a wired or wireless connection, accessing the neural signal data from a memory, or the like.

The duration of the learning is adjustable by the user and can be tailored to meet an assumption of ergodicity. As a non-limiting example, the duration of the learning can be on the order of three minutes, which has been observed to be a reasonable default for most brain areas and oscillatory signals. Next, the user specifies the region-of-interest, the desired frequency band, and the desired phase. Note that the technical delay of the system should also be provided (e.g., hardware and software delays, which are system dependent). This delay can be measured beforehand and allows the global optimization-based temporal prediction framework to account for system-specific delays and provide high timing accuracy in stimulus delivery. Then, neural signal data are analyzed to extract the optimal parameters (e.g., by applying spatial filter, power cutoff, projection distance, window length, padding length, length of edge removal, projection starting point) for phase prediction. To do this, these steps can be implemented.

For one or more locations of interest, spatial filtering can be performed by calculating the higher-order derivative of the instantaneous spatial voltage distribution from all sensors. This step provides spatial filtering, which accentuates localized neural signal in the radial source proximal to the electrode/sensor and reduces global and diffused activity.

In order to predict the phase accurately, it is advantageous to have a high-quality signal that is not contaminated by noise. Because it is challenging to fully eliminate noise, a threshold for signal power can be defined that ensures that the neural signal data used for phase projection have a high signal-to-noise ratio (i.e., is strong enough relative to noise). For this, after applying the spatial filtering to the acquired recording, the neural signal can be split into shorter trials, and the signal power can be measured in the frequency band of interest for each trial. Then, a threshold is defined based on these power values. In the phase prediction, the trials whose power is below this threshold are excluded.

The ground truth can be calculated as the benchmark for the accuracy of phase prediction. As a non-limiting example, this can be done by acausal, or non-causal, filtering (i.e., to avoid the limitations of causal signal processing imposed in real-time processing) of the neural signal in the frequency band of interest, and analyzing the signal phase at all time points using the Hilbert transform, or the like. The ground-truth phase can be used to compare with the predicted phase in the next step.

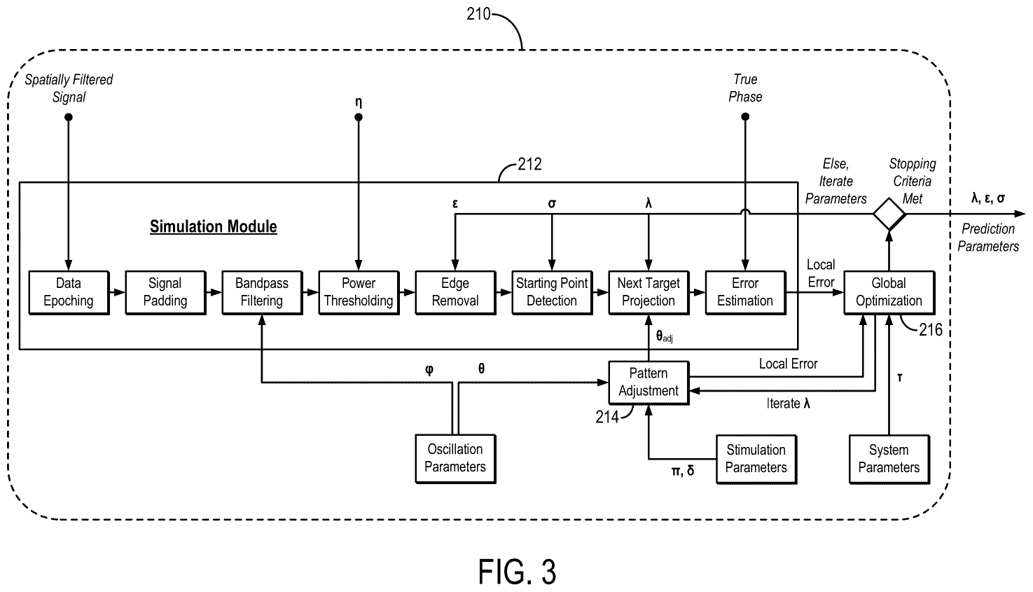

An automatic module 210 is defined to find the best prediction parameters (e.g., projection distance, window length, padding length, length of edge removal, projection starting point). As shown in FIG. 3, the automatic module 210 can include a simulation module 212, a pattern adjustment process module 214, and a global optimization process module 216. The simulation module 212 is generally designed to quantify the accuracy of phase prediction for any given set of prediction parameters to be used in the global optimization process, which may implement a Bayesian optimization or other suitable global optimization paradigm. To this end, the simulation module 212 splits the ergodic signal recorded previously (e.g., in the learning module 120) into trials with an input window length, pads the signal in each trial (e.g., with an input pad length on each side), bandpass filters (e.g., brickwall filter) the signal in the frequency band of interest, checks the power of signal, and removes the edge of the signal based on the input parameter. Then, the location of the last peak or trough (depending on the projection starting point parameter) is detected in each trial. The next desired phase is then estimated by projecting the peak/trough by the projection distance provided as the input, and the actual predicted phase for each trial is computed from the ground truth phase at the projected timepoint. By comparing the actual predicated phase and the desired phase, a distribution of the deviations can be measured across trials. Based on this distribution, the performance error given the input parameters can be quantified as,

$$Local error = \frac{1}{π N} {\sum }_{i = 1}^{N} \vert {θ}_{i} - {θ}_{0} \vert ; \qquad ( 1 )$$

where N is the number of trials for which the phase is estimated, θᵢ is the estimated phase (in radians) for the ith trial, and θ₀ (rad) is the desired phase. A local error of zero means that the phase has been estimated precisely at every trial.

The optimal parameters (e.g., projection distance, window length, padding length, length of edge removal, projection starting point) are automatically determined by using a global optimization method (as shown in FIG. 3 and discussed in more detail below). In this approach, the set of parameters that would minimize the local error as the output of the module discussed above is determined. These parameters are then used in the online prediction module 140.

If the user is interested in delivering a patterned train of stimuli instead of a single stimulus at the desired time, an additional step can be applied between the steps of calculating the ground truth and quantifying the accuracy of the phase prediction outlined above. For this, to ensure the center of the stimulus train falls on the desired phase, the targeted phase is adjusted to correspond to the beginning of the train. The following equation accounts for this phase adjustment:

$$TL = \frac{( NS - 1 ) \times ISI \times fs}{1000} ; \qquad ( 2 )$$

where TL is the train length (e.g., samples), NS is the desired number of stimuli, ISI is the user-specified inter-stimulus interval (ms), and fs is the sampling frequency (Hz). The phase adjustment can then be computed as,

$${θ}_{adj} = {θ}_{0} - ⌊ \frac{π TL}{CL} ⌉ ; \qquad ( 3 )$$

where θ_{adj} and θ₀ (rad) are the adjusted and original desired phase, respectively. The operator “[ . . . ]” rounds the number to the nearest integer. TL is the train length calculated above and CL is the cycle length (samples) in the learning data for the frequency band of interest. CL can be calculated similar to the steps outlined above to calculate the projection distance from the peak to the next peak.

As noted above, the global optimization process module 216 is used to automatically determine optimal parameters. The global optimization process module 216 can implement a Bayesian optimization. Alternatively, other global optimization paradigms can also be implemented. As a non-limiting example, Bayesian optimization can be used to find the set of hyperparameters that would minimize the following error function:

$$\hat{x} = {arg min}_{x ∈ χ} ( f ( x ) ) ; \qquad ( 4 )$$

where \hat{x} is the vector of optimal parameters, f is the error function (local error from the simulation module 212), and χ is the domain bounding the parameters.

First, a Gaussian process model of ƒ(x) is created:

$$f ( x ) = GP ( μ ( x ) , k ( x , {x}^{′} ) ) ; \qquad ( 5 )$$

where GP is the Gaussian process function, μ(x) is the mean function (prior mean is set to 0), and k(x, x′) is the covariance kernel (function) at the predictor value of x and x′ which is defined as,

$$k ( x , {x}^{′} ) = {σ}_{f}^{2} ( 1 + \frac{\sqrt{5} r}{{σ}_{ℓ}} + \frac{5 {r}^{2}}{3 {σ}_{ℓ}^{2}} ) exp ( - \frac{\sqrt{5} r}{{σ}_{ℓ}} ) ; \qquad ( 6 )$$

where σ_{ƒ} is the signal standard deviation, σₗ is the characteristic length scale, and r is the Euclidian distance between x and x′:

$$r = \sqrt{{( x - {x}^{′} )}^{T} ( x - {x}^{′} )} . \qquad ( 7 )$$

Given the observations at the previous t points, f=[ƒ(x₁), ƒ(x₂), . . . , ƒ(xₜ)], we can make a prediction about the function value at a new point ƒ*=ƒ(x*) using:

$$Pr ( {f}^{*} \vert f ) = N ( μ ( {x}^{*} ) , σ ( {x}^{*} ) ) ; \qquad ( 8 )$$

where N is the normal distribution with mean and variance of:

$$μ ( {x}^{*} ) = K ( {x}^{*} , X ) {K ( X , X )}^{- 1} f ; \qquad ( 9 )$$

$${σ}^{2} ( {x}^{*} ) = K ( {x}^{*} , {x}^{*} ) - K ( {x}^{*} , X ) {K ( X , X )}^{- 1} K ( X , {x}^{*} ) ; \qquad ( 10 )$$

where K(X,X) is t×t matrix where element (i,j) is given by k(xᵢ, xⱼ), and K(X, x*) is t×1 vector where element i is given by k(xᵢ, x*) from the covariance kernel in Eqn. (6).

Based on the predicted model ƒ and the associated probabilities, the next point to sample is determined. This point can be selected by using an acquisition function to ensure the best trade-off between exploration and exploitation is found. As a non-limiting example, the expected improvement method can be used for the acquisition function,

$$EI ( {x}^{*} ) = {\int }_{∞}^{f ( {x}_{best} )} ( f ( {x}_{best} ) - f ( {x}^{*} ) ) N ( μ ( {x}^{*} ) , σ ( {x}^{*} ) ) df ( {x}^{*} ) ; \qquad ( 11 )$$

where x_{best} is the vector of parameters corresponding to the current minimum. The next point is selected as the x that maximizes the acquisition function.

In order to obtain better improvement per second, time-weighting can be used in the acquisition function:

$$EIpS ( {x}^{*} ) = \frac{EI ( {x}^{*} )}{{μ}_{S} ( {x}^{*} )} ; \qquad ( 12 )$$

where μ_{S}(x*) is the posterior mean of the timing Gaussian process model.

The sampling is iteratively repeated using the steps above until the stopping criteria is met or the maximum objective evaluations (e.g., 50, 100, etc.) is reached.

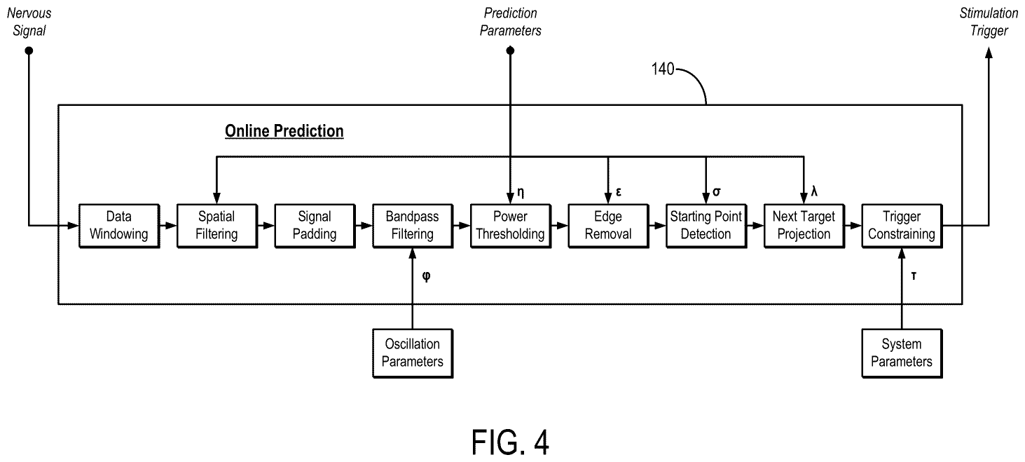

During the online prediction, the data are streamed to the processor in real-time, and the signal data are analyzed continuously to estimate the time of the next desired phase, as illustrated in FIG. 4. The procedure for phase detection is similar to the phase detection step used in the simulation module 212, but without predicting the accuracy by comparing to the ground truth. Then, based on the phase detection and other stimulation constraints (e.g., minimum inter-stimulus interval), a stimulus (or a train of stimuli) is delivered at the appropriate time, accounting for stimulator hardware delay.

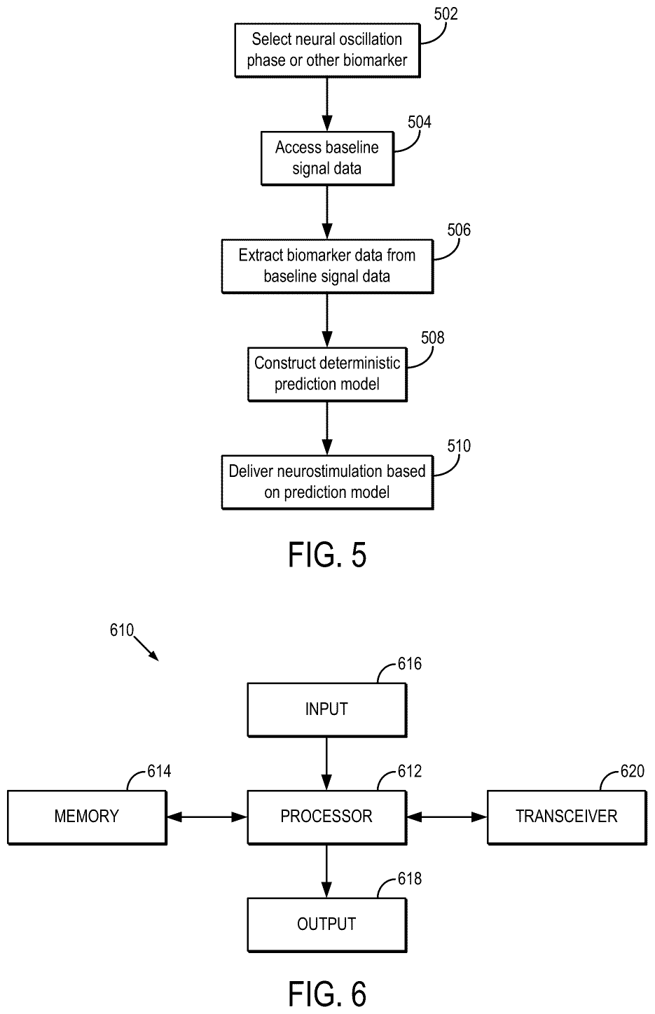

Referring now to FIG. 5, a flowchart is illustrated as setting forth the steps of an example method for controlling a closed-loop brain stimulation device using a global optimization-based temporal prediction framework.

A biomarker at which stimulation should be applied is selected or otherwise set by a user, as indicated at step 502. For example, the biomarker can correspond to a neural oscillation phase, such as a brain oscillation phase. The neural oscillation phase can be selected or otherwise set as described above. Additionally, other parameters can be set by a user. For instance, the user can define an area of interest (e.g., based on monitoring electrode(s)), the neural process of interest (e.g., frequency range of the brain oscillation or other neural oscillation of interest), and the type of stimulation that should be applied (e.g., a single pulse or any pattern of pulses or other stimulation treatments).

Baseline signal data are then accessed with a computer system, which may be a controller of a brain stimulation device or a separate computer system or processor, as indicated at step 504. Accessing the baseline signal data can include retrieving previously recorded baseline signal data from a memory or other data storage device or medium. Additionally or alternatively, accessing the baseline signal data can include recording the baseline signal data and communicating those data to the controller.

Biomarker data (e.g., neural oscillation phase data) are the extracted from the baseline signal data, as indicated at step 506. For example, the baseline signal data can be provided to a learning module 120 of a global optimization-based temporal prediction framework. A deterministic prediction model of biomarker (e.g., neural oscillation phase) occurrences is then constructed, as indicated at step 508. This model can be constructed and its hyperparameters can be determined or otherwise estimated using global optimization (e.g., Bayesian optimization), as described above in detail. As described above, the model can be used to determine or otherwise predict the timing of an occurrence of the biomarker (e.g., a neural oscillation phase occurrence). Using this model, neurostimulation can then be delivered to the subject, as indicated at step 510. For instance, neural signal data can be measured in real-time from the subject and applied to the constructed model. In doing so, control parameter settings are generated, which may be sent to the neurostimulation device to control its operation.

Referring now to FIG. 6, an example of a controller 610 that can implement the methods described in the present disclosure to control a neurostimulation device, such as a brain stimulation device, is illustrated. In general, the controller 610 includes a processor 612, a memory 614, and input 616, and an output 618. The controller 610 can be implemented as part of a neurostimulation device, or as a separate controller that is in communication with the neurostimulation device via the output 618. As one example, the controller 610 can be implemented in a neurostimulation device, such as an implantable medical device (e.g., an implanted nerve stimulation system), a standalone neurostimulation device (e.g., an externally applied electrical, magnetic, sonic, and/or thermal stimulation device), and so on. In other examples, the controller 610 can be implemented in a remote computer that communicates with the neurostimulation device. In still other examples, the controller 610 can be implemented in a smartphone that is paired with the neurostimulation device, such as via Bluetooth or another wireless or wired communication.

In some embodiments, the input 616 is capable of recording neural signal data, or other physiological measurement data, from the user. As one example, the neural signal data can be electrophysiological activity data (e.g., EEG signal data), and the input 616 can be one or more electrodes. The input 616 can include a wired or wireless connector for receiving neural signal data. These neural signal data can be transmitted to the controller 610 via the input 616.

The processor 612 includes at least one hardware processor to execute instructions embedded in or otherwise stored on the memory 614 to implement the methods described in the present disclosure. The memory can also store baseline signal data, measured neural signal data, one or more models, and model parameters, as well as settings to be provided to the processor 612 for generating control signals to be provided to a neurostimulation device via the output 618.

The output 618 communicates control signals to a neurostimulation device, which may be a brain stimulation device such as a TMS device, or another neurostimulation device (e.g., another electrical, magnetic, sonic, and/or thermal stimulation device). As one example, where the neurostimulation device is a TMS device, the control signals provided to the output 618 can control one or more electromagnetic coils to operate under control of the controller 610 to deliver electromagnetic stimulations (e.g., by generating magnetic fields) to the subject. Circuitry in the controller 610 can detect and processes electrophysiological activity sensed by the one or more electrodes via the input 616 to determine the optimized stimulation settings (e.g., timing when to apply stimulation) based on the methods and algorithms described above. The optimized settings are provided as instructions to a pulse generator in the neurostimulation device via the output 618, which in response to the instructions provides an electrical signal to the one or more electrodes or electromagnetic coils to deliver the neurostimulations to the subject.

The controller 610 can also include a transceiver 620 and associated circuitry for communicating with a programmer or other external or internal device. As one example, the transceiver 620 can include a telemetry coil. In some embodiments, the transceiver 620 can be a part of the input 616.

In operation, the controller 610 receives neural signal data from the subject via the input 616. These neural signal data are provided to the processor 612 where they are processed. For example, the processor 612 analyzes the neural signal data using a global optimization-based temporal prediction framework in order to determine optimal settings for the neurostimulation device.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), Flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for controlling a neurostimulation device, the method comprising:
(a) selecting, by a computer system, a biomarker for controlling a delivery of neurostimulation with a neurostimulation device;
(b) accessing baseline neural signal data with the computer system, wherein the baseline neural signal data have been acquired from a nervous system of a subject;
(c) processing the baseline neural signal data with the computer system to extract features from the baseline neural signal data corresponding to the selected biomarker;
(d) constructing, with the computer system, a predictive model of biomarker occurrences based on the extracted features;
(e) receiving neural signal data with the computer system in real-time; and
(f) controlling operation of the neurostimulation device by applying the neural signal data to the predictive model of biomarker occurrences, generating output as stimulation parameters that indicate time points at which to deliver neurostimulation, wherein the time points correspond to predicted occurrences of the biomarkers within the neural signal data.

2. The method of claim 1, wherein the biomarker comprises a neural oscillation phase.

3. The method of claim 2, wherein the neural oscillation phase corresponds to a phase of at least one frequency band in the neural signal data.

4. The method of claim 3, wherein the neural oscillation phase corresponds to a brain oscillation phase, and the frequency band corresponds to at least one of an alpha oscillation phase, a beta oscillation phase, a gamma oscillation phase, a delta oscillation phase, or a theta oscillation phase.

5. The method of claim 1, wherein constructing the predictive model comprises inputting the baseline neural signal data to a learning module implemented with the computer system, generating output as the predictive model.

6. The method of claim 5, wherein the learning module implements a global optimization to determine a set of prediction parameters that minimizes local errors in predicting the biomarker.

7. The method of claim 6, wherein the global optimization is implemented as a Bayesian optimization.

8. The method of claim 7, wherein constructing the predictive model further comprising tuning hyperparameters of the predictive model based on the Bayesian optimization.

9. The method of claim 6, wherein constructing the predictive model further comprises tuning hyperparameters of the predictive model based on the global optimization.

10. The method of claim 1, wherein the neurostimulation device comprises at least one of a magnetic stimulation device, an electric stimulation device, a sonic stimulation device, or a thermal stimulation device.

11. The method of claim 10, wherein the neurostimulation device comprises a transcranial magnetic stimulation device.

12. A non-transitory computer-readable storage medium having stored thereon instructions that when executed by a processor cause the processor to:
select a biomarker;
receive baseline neural signal data that have been acquired from a nervous system of a subject;
process the baseline neural signal data to extract features from the baseline neural signal data corresponding to the selected biomarker;
construct a predictive model of biomarker occurrences based on the extracted features;
receiving neural signal data in real-time from the subject;
generate control parameter settings by applying the neural signal data to the predictive model of biomarker occurrences, wherein the control parameter settings indicate time points at which to deliver neurostimulation, the time points corresponding to predicted occurrences of the biomarkers within the neural signal data; and
send the neurostimulation device control parameter settings to a neurostimulation device.

13. The non-transitory computer-readable storage medium of claim 12, wherein the biomarker comprises a neural oscillation phase.

14. The non-transitory computer-readable storage medium of claim 13, wherein the neural oscillation phase corresponds to a phase of at least one frequency band in the neural signal data.

15. The non-transitory computer-readable storage medium of claim 14, wherein the neural oscillation phase corresponds to a brain oscillation phase, and the frequency band corresponds to at least one of an alpha oscillation phase, a beta oscillation phase, a gamma oscillation phase, a delta oscillation phase, or a theta oscillation phase.

16. The non-transitory computer-readable storage medium of claim 12, wherein constructing the predictive model comprises inputting the baseline neural signal data to a global optimization to determine a set of prediction parameters that minimizes local errors in predicting the biomarker.

17. The non-transitory computer-readable storage medium of claim 16, wherein the global optimization is implemented as a Bayesian optimization.

18. The non-transitory computer-readable storage medium of claim 17, wherein constructing the predictive model comprises tuning hyperparameters of the predictive model based on the Bayesian optimization.

19. The non-transitory computer-readable storage medium of claim 16, wherein constructing the predictive model comprises tuning hyperparameters of the predictive model based on the global optimization.
